(19) United States
(12) Patent Application Publication
(10) Pub. No.: US 2024/0226086 A1
(43) Pub. Date: Jul. 11, 2024

(54) LIQUID PHARMACEUTICAL FORMULATIONS OF QUINOLINES

(71) Applicant: TaP Pharmaceuticals, AG, Baar (CH)

(72) Inventors: Nilesh PARIKH, Irvine, CA (US); William HITE, Winchester, CA (US)

(73) Assignee: TaP Pharmaceuticals, AG, Baar (CH)

(21) Appl. No.: 18/091,053

(22) Filed: Dec. 29, 2022

(51) Int. Cl. A61K 31/4706; A61K 9/00; A61K 9/08; A61K 47/10; A61P 33/06

(52) U.S. Cl. CPC A61K 31/4706; A61K 9/0053; A61K 9/08; A61K 47/10; A61P 33/06

(57) ABSTRACT

Certain embodiments of the present disclosure provide liquid pharmaceutical formulations, suitable for oral administration, that contain from 1 mg/ml to 300 mg/ml of chloroquine, hydroxychloroquine, or pharmaceutically acceptable salt(s) thereof and a solvent that is one or more of a glycerin, a propylene glycol, and a polyethylene glycol. Such formulations are free of added water and contain an amount of the the solvent is present sufficient to result in the chloroquine, hydroxychloroquine, or pharmaceutically acceptable salt thereof being in the solution phase of the formulation.

### FIELD

The instant disclosure provides liquid pharmaceutical formulations, suitable for oral administration, that comprise a cinchona alkaloid or quinoline drug and that exhibit advantageous solubility properties, together with methods of making and using same.

### BACKGROUND

Some report that the discovery of quinine as the most serendipitous medical discovery of the 17th century and that the treatment of malaria with quinine marked the first successful use of a chemical compound to treat an infectious disease. Quinine, as a component of the bark of the cinchona (quina-quina) tree, was used to treat malaria from as early as the 1600s, when it was referred to as the “Jesuits' bark,” “cardinal's bark,” or “sacred bark.” (See, e.g., Achan et al. Malaria Journal 2011, 10:144.) Quinine is a quinoline, and the chemical structure of quinine is:

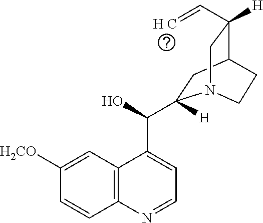

Other quinolines have efficacy in treating malaria include quinidine, cinchonine, dihydroquinine, dihydroquinidine, cinchonidine, chloroquine, and hydroxychloroquine. (See, e.g., Achan et al. Malaria Journal 2011, 10:144.)

Some report that quinine was not chemically synthesized until 1944, and there has never been a synthesis that can compete with extraction from its bark. Bayer had started a program to test the company's synthetic compounds for efficacy in treating malaria activity and to make analogs around the active ones. That led in 1934 to the synthesis of chloroquine by Hans Andersag at Bayer. (See, e.g., Lowe, Chloroquine, Past and Present. Science: 20 Mar. 2020) The chemical structure of chloroquine is:

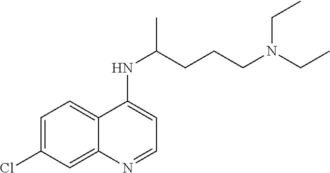 [SMILES: CCN(CC)CCCC(C)Nc1ccnc2cc(Cl)ccc12]

Chloroquine (“CQ”) was and continues to be used for the treatment of malaria and is on the World Health Organization's list of essential medicines.

Hydroxychloroquine (“HCQ”) was approved for medical use in the United States in 1955 and is currently on the World Health Organization's list of essential medicines. HCQ is the active pharmaceutical ingredient in PLAQUENIL®, which is a tablet containing 200 mg HCQ and indicated for the treatment of uncomplicated malaria due to Plasmodium falciparum, rheumatoid arthritis, lupus erythematosus, and chronic discoid lupus erythematosus. PLAQUENIL® is also indicated for the pprophylaxis of malaria in geographic areas where chloroquine resistance is not reported. HCQ is chemically described as 2-[[4-[(7-Chloro-4-quinolyl) amino]pentyl] ethylamino] ethanol sulfate (1:1) with the molecular formula C₁₈H₂₆ClN₃O·H₂SO. The chemical structure of HCQ is:

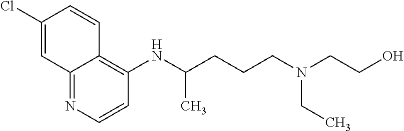 [SMILES: CCN(CCO)CCCC(C)Nc1ccnc2cc(Cl)ccc12]

A side-by-side depiction of CQ, HCQ, and quinine is:

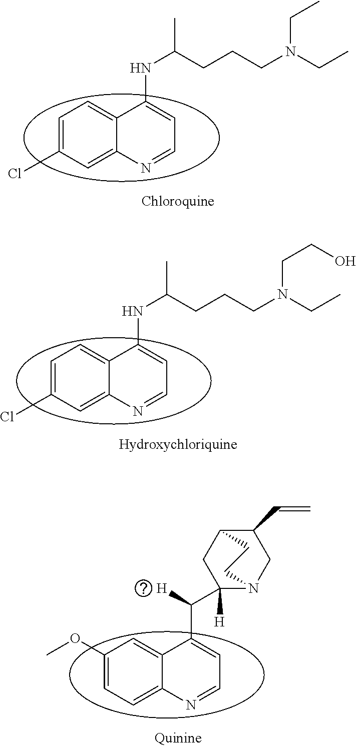

Patent Cooperation Treaty Patent Application Publication WO 2021/259673 (the “'673 publication”) describes HCQ's mechanism of action as being complex. The '673 publication recites that HCQ decreases pro-inflammatory cytokine secretion, impairs immune cell function, reverses platelet activation induced by human antiphospholipid antibodies, protects the annexin A5 anticoagulant shield from disruption by antiphospholipid antibodies, and markedly suppresses the TLR9-mediated human B cell functions during inflammatory processes.

Perrone et al. discloses that HCQ tablets cannot be administered to non-cooperative patients, such as those in intensive care units or, more in general, unable to swallow solid dosage forms. Perrone et al. further discloses that the hospital pharmacist must manipulate the solid dosage form to prepare an HCQ suspension, even if it can strongly affect the product quality. (Perrone et al. Stability data of extemporaneous suspensions of hydroxychloroquine sulphate in oral liquid bases after tablet manipulation. Science Direct, Data in Brief, Volume 33, December 2020, 106575.) Similarly, the '673 publication recites that children under six years old and geriatric populations have difficulties in swallowing tablets. For those populations, the tablets are crushed to a powder that is then dissolved in some beverage such as fruit juice or water and administered to the patient. The '673 publication recites that this practice leads to major problems concerning dosage accuracy and thus the drug efficacy.

Pharmaceutical Technology describes liquid drug formulations as containing, in addition to active drug substances, excipients that serve different and specialized pharmaceutical purposes in the formulation. (Pharmaceutical Technology. Excipients used in the formulation of lquid dosage forms. Pharmapproach Ltd, Pharmaceutics. Oct. 11, 2020. The excipients typically form the bulk of liquid formulations, making it essential that excipients in liquid drug formulations are physically and chemically compatible with the drug substance and each other. Pharmaceutical Technology reports several categories of excipients, including vehicles/solvents, co-solvents, surfactants, preservatives, viscosity modifiers/suspending agents, buffers, antioxidants, chelating agents, sweeteners, flavoring agents, colorants, antifoaming agents, humectants, emulsifying agents, and flocculating agents. The reference treats “vehicle” and “solvent” as interchangeable terms meaning the major components used as a base in which drugs and other excipients are dissolved or dispersed. It teaches that two broad categories of vehicles/solvents used in liquid drug formulations are aqueous (e.g., water, polyhydric alcohols, hydro-alcoholic solutions, and buffers) and oily (e.g., vegetable or mineral oils, organic oily bases, and emulsified bases). The choice of vehicle/solvent used depends on the nature and physicochemical properties of the drug substance and the intended use of the formulation. Pharmaceutical Technology surveys several particular vehicles/solvents, namely: water, alcohol, glycerol, propylene glycol, and polyethylene glycol (PEG). It describes glycerol as a triol alcohol (without the central nervous system depressant activity of ethanol) that is a clear, colorless, odorless, viscous, hygroscopic liquid with sweet taste, approximately 0.6 times as sweet as sucrose. It reports that glycerol is miscible with water, alcohol, propylene glycol, and polyethylene glycol 400. As a solvent, the solubilizing properties of glycerol are comparable to alcohol but because of its viscosity, solutes are slowly soluble in it unless it is rendered less viscous by heating. Pharmaceutical Technology also reports that the increased viscosity imparted to the final product may be an undesired outcome of glycerol's use.

Pharmaceutical Technology teaches that alcohol, frequently referred to as ethyl alcohol or ethanol, is the most commonly used solvent in liquid pharmaceutical formulation next to water. It is a clear, colorless, mobile, and volatile liquid with a slight, characteristic odor and burning taste. Alcohol USP contains ethanol, C₂H₅OH, not less than 92.3% and not more than 93.8%, by weight, which corresponds to not less than 94.9% and not more than 96.0%, by volume. Ethanol is miscible with water, glycerine, propylene glycol, and polyethylene glycol 400. It is used as a primary solvent for many organic compounds. Water-alcohol mixtures can be very effective in solubilizing poorly soluble drugs.

As indicated in Table A, prior art references report that HCQ is either soluble in water (87 mg/ml) or very slightly soluble in water (0.18 mg/ml) and insoluble, practically insoluble, very slightly soluble, or sparingly soluble in several other solvents, including glycerol. (See, e.g., Cayman Chemical, Product Information Hydroxychloroquine Sulfate. Oct. 11, 2022; Seleckchem.com, Hydroxychloroquine Sulfate (NSC 4375) Datasheet. Dec. 27, 2022; ALKALOIDA Chemical Company Zrt, HYDROXYCHLOROQUINE SULFATE TABLETS, USP, package insert. August, 2018; and Agnihotrik et al., Formal chemical stability analysis and solubility analysis of artesunate and hydroxychloroquinine for development of parenteral dosage form. Journal of Pharmacy Research: (6:1), January 2013, Pages 117-122.)

TABLE A
Solubility of hydroxychloroquine in various solvents

| Solvent | Inference as USP |
|---|---|
| Alcohol | Practically insoluble or slightly soluble |
| Chloroform | Practically insoluble or slightly soluble |
| Dimethylformamide | Sparingly soluble |
| Dimethylsulfoxide | Insoluble or sparingly soluble |
| Ethanol | Sparingly soluble or insoluble |
| Ether | Practically insoluble or very slightly soluble |
| Glycerol | Very slightly soluble |

Table B reports definitions for solubility classifications in the pharmaceutical field. (See, O. Wolk, et al. Provisional in-silico biopharmaceutics classification (BCS) to guide oral drug product development. Drug design, development and therapy. 2014:8. 1563-75. 10.2147/DDDT.S68909.)

TABLE B
Solubility Classifications

| | Parts of solvent | Solubility | Solubility |
|---|---|---|---|
| Descriptive term | required for | Range | Assigned |
| (solubility definition) | one part of solute | (mg/ml) | (mg/ml) |
| Very soluble | <1 | ≥1,000 | 1,000 |
| Freely soluble | From 1 to 10 | 100-1,000 | 100 |
| Soluble | From 10 to 30 | 33-100 | 33 |
| Sparingly soluble | From 30 to 100 | 10-33 | 10 |
| Slightly soluble | From 100 to 1,000 | 1-10 | 1 |
| Very slightly soluble | From 1,000 to 10,000 | 0.1-1 | 0.1 |
| Practically insoluble | ≥10,000 | <0.1 | 0.01 |

### SUMMARY

Certain embodiments of the present disclosure provide liquid pharmaceutical formulations, suitable for oral administration, that comprise a therapeutically effective amount of an active pharmaceutical ingredient (“API”) that is one or more of a cinchona alkaloid or a quinoline such as chloroquine, hydroxychloroquine, or pharmaceutically acceptable salt(s) thereof; and an amount of a solvent sufficient to result in from more than 1 mg/ml, for example from 2 to 300 mg/ml of the cinchona alkaloid or the quinoline such as chloroquine, hydroxychloroquine, or the pharmaceutically acceptable salt(s) thereof being in the solution phase of the formulation, when the formulation is kept at 25° C.±2° C. In such formulations, the solvent is one or more of glycerol and a polyethylene glycol, and such formulations are free of added water. In some of such formulations, the API is HCQ sulfate, of which from 10 mg/ml to 275 mg/ml, from 25 mg/ml to 275 mg/ml, from 40 mg/ml to 50 mg/ml, 40 mg/ml, 45 mg/ml, 50 mg/ml, or 100 mg/ml is in the solution phase of the formulation. Also in some of such formulations, the solvent is glycerol, in an amount of from 630 mg/ml to 1260 mg/ml, from 462.5 mg/ml to 1260 mg/ml, from 945 mg/ml to 1260 mg/ml, 1260 mg/ml, or balance glycerol. Also in some of such formulations, the solvent consists of glycerol.

By “free of added water” is meant that no water is intentionally added to the formulation. Notwithstanding that no water is intentionally added, formulations as disclosed herein can sometimes contain trace amounts, or even significant amounts (e.g. up to 0.1%, or up to 0.5%, or up to 1%, or up to 2%, or up to 5%), of water due to, e.g. hydrate forms of the API used in the formulation, or hygroscopicity of one or more of the ingredients of the formulation.

Certain embodiments of the present disclosure provide liquid pharmaceutical formulations, suitable for oral administration, that comprise a therapeutically effective amount of an API that is one or more of CQ, HCQ, or pharmaceutically acceptable salt(s) thereof, preferably HCQ sulfate; and an amount of a solvent sufficient to result in from more than 1 mg/ml, for example, 2 to 300 mg/ml of the chloroquine, the HCQ, or the pharmaceutically acceptable salt(s) thereof being in the solution phase of the formulation, when the formulation is kept at 25° C.±2° C. In such formulations, the solvent can be propylene glycol, and such formulations are free of added water. In some of such formulations, from 10 mg/ml to 275 mg/ml, from 25 mg/ml to 275 mg/ml, from 40 mg/ml to 50 mg/ml, 40 mg/ml, 45 mg/ml, 50 mg/ml, or 100 mg/ml of the HCQ is in the solution phase of the formulation. Also in some of such formulations, the concentration of propylene glycol is from 500 mg/ml to 1050 mg/ml, from 750 mg/ml to 1050 mg/ml, from 1000 mg/ml to 1040 mg/ml, 1040 mg/ml, or balance propylene glycol.

Certain embodiments of the present disclosure provide liquid pharmaceutical formulations, suitable for oral administration, that comprise a therapeutically effective amount of an API that is one or more of CQ, HCQ, or pharmaceutically acceptable salt(s) thereof, preferably HCQ sulfate; and an amount of a solvent sufficient to result in from more than 1 mg/ml, for instance from 2 to 300 mg/ml of the chloroquine, the HCQ, or the pharmaceutically acceptable salt(s) thereof being in the solution phase of the formulation, when the formulation is kept at 25° C.±2° C. In such formulations, the solvent can be a polyethylene glycol, and such formulations are free of added water. In some of such formulations, from 10 mg/ml to 275 mg/ml, from 25 mg/ml to 275 mg/ml, from 40 mg/ml to 50 mg/ml, 40 mg/ml, 45 mg/ml, 50 mg/ml, or 100 mg/ml of the HCQ is in the solution phase of the formulation. Also in some of such formulations, the concentration of polyethylene glycol is from 565 mg/ml to 1130 mg/ml, from 847.5 mg/ml to 1130 mg/ml, from 1000 mg/ml to 1128 mg/ml, 1128 mg/ml, or balance polyethylene glycol.

Certain embodiments of the present disclosure provide methods of treating uncomplicated malaria due to Plasmodium falciparum, rheumatoid arthritis, lupus erythematosus, or chronic discoid lupus erythematosus, comprising orally administering a formulation of the disclosure to a subject presenting treating uncomplicated malaria due to Plasmodium falciparum, rheumatoid arthritis, lupus erythematosus, or chronic discoid lupus erythematosus. In some of such methods, the formulation is administered to the subject once daily and the formulation contains from 40 mg/ml to 100 mg/ml, 40 mg/ml, 45 mg/ml, 50 mg/ml, or 100 mg/ml of the chloroquine, HCQ, or pharmaceutically acceptable salt(s) thereof of.

Certain embodiments of the present disclosure provide liquid pharmaceutical formulations, suitable for oral administration, that comprise 40 mg/ml, 45 mg/ml, 50 mg/ml, or 100 mg/ml HCQ sulfate; and at least 945 mg/ml glycerol or balance glycerol. Such formulations were free of added water and contained 95%, 96%, 97%, 98%, 99% or more of the HCQ sulfate in their solution phase, when the formulation is kept at 25° C.±2° C.

Certain embodiments of the present disclosure provide a process for preparing a liquid pharmaceutical formulation as described above. Such a process can be one comprising:
(i) forming an API-solvent mixture by combining a therapeutically effective amount of an API and an amount of a solvent sufficient to result in from 2 mg/ml to 300 mg/ml of the API being in the solution phase of the liquid formulation, wherein the API is CQ, HCQ, or a combination thereof, or one or more pharmaceutically acceptable salt(s) thereof and wherein the solvent is glycerol, a PEG, or a combination thereof; and
(ii) holding the temperature of the API-solvent mixture at a range of from 25° C. to 290° C., or at a temperature of 20° C., 25° C., 30° C., 35° C., 40° ° C., 45° C., 50° C., 55° C., 60° C., 65° ° C., 70° C., 75° C., 80° C., 85° C., 90° C., 95° C., 100° ° C., 110° ° C., 125° C., 150° C., 175° C., 200° C., 150° C., 200° C., 150° C., 275° C., or 100° C., or range between any two of the stated temperatures, until from 2 mg/ml to 300 mg/ml of the API is in the solution phase of the mixture and thereby preparing the liquid formulation; and
(iii) cooling, actively or passively, the temperature of the liquid to at 25° C.±2° C. at a rate that maintains the 2 mg/ml to 300 mg/ml of the API being in the solution phase of the liquid formulation
wherein the liquid formulation is free of added water.

### DETAILED DESCRIPTION

The present disclosure provides liquid pharmaceutical formulations of an active pharmaceutical ingredient (API) that is a cinchona alkaloid and/or a quinoline such as chloroquine and/or hydroxychloroquine that are suitable for oral administration. These formulations exhibit surprisingly advantageous high levels of dissolved API, or pharmaceutically acceptable salts thereof, solubilities not attainable prior to the instant disclosure. Exemplary cinchona alkaloids useful in liquid formulations of the present disclosure include, without limitation, quinine, quinidine, cinchonine, dihydroquinine, dihydroquinidine, cinchonidine, quinolone, and hydroxyquinoline. Such formulations of the disclosure are useful for treating uncomplicated malaria due to Plasmodium falciparum, rheumatoid arthritis, lupus erythematosus, and chronic discoid lupus erythematosus and for the prophylaxis of malaria in geographic areas where chloroquine resistance is not reported.

Formulations as disclosed herein can “comprise” a list of ingredients, such list then being open to inclusion of further unspecified ingredients. Alternatively, formulations as disclosed herein can “consist of” a list of ingredients, meaning that the formulations include only the listed ingredients. Or, formulations as disclosed herein can “consist essentially of” the listed ingredients, meaning that the formulations include all of the listed ingredients, and may include as well any further ingredients that do not materially affect the utility of the formulation. For purposes of the present disclosure, that utility is a liquid pharmaceutical formulation having a high concentration of a cinchona alkaloid or quinolone (e.g. CQ or HCQ) API.

In some embodiments, formulations of the present disclosure contain the cinchona alkaloid and/or the quinoline such as CQ or HCQ, or pharmaceutically acceptable salt thereof as an API, at concentrations in the formulations overall, or in the solution phase of the formulations, of from 1 mg/ml to 500 mg/ml and further exemplary concentrations include 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 12.5 mg/ml, 15 mg/ml, 17.5 mg/ml, 20 mg/ml, 22.5 mg/ml, 25 mg/ml, 27.5 mg/ml, 30 mg/ml, 32.5 mg/ml, 35 mg/ml, 37.5 mg/ml, 40 mg/ml, 42.5 mg/ml, 45 mg/ml, 47.5 mg/ml, 50 mg/ml, 52.5 mg/ml, 55 mg/ml, 57.5 mg/ml, 60 mg/ml, 62.5 mg/ml, 65 mg/ml, 67.5 mg/ml, 70 mg/ml, 72.5 mg/ml, 75 mg/ml, 77.5 mg/ml, 80 mg/ml, 82.5 mg/ml, 85 mg/ml, 87.5 mg/ml, 90 mg/ml, 92.5 mg/ml, 95 mg/ml, 97.5 mg/ml, 100 mg/ml, 125 mg/ml, 150 mg/ml, 175 mg/ml, 200 mg/ml, 225 mg/ml, 250 mg/ml, 275 mg/ml, 300 mg/ml, 325 mg/ml, 350 mg/ml, 375 mg/ml, 400 mg/ml, 425 mg/ml, 450 mg/ml, 475 mg/ml, and 500 mg/ml, as well as in a range between any two of said API concentrations.

In some embodiments, formulations of the present disclosure contain cinchona alkaloid or quinoline, e.g., CQ or HCQ, or pharmaceutically acceptable salt thereof as an API, in weight to volume proportions of from 0.5% w/v to 20% w/v and exemplary particular weight to volume proportions include 0.5% w/v, 1% w/v, 1.25% w/v, 1.5% w/v, 1.75% w/v, 2% w/v, 2.25% w/v, 2.5% w/v, 2.75% w/v, 3% w/v, 3.25% w/v, 3.5% w/v, 3.75% w/v, 4% w/v, 4.25% w/v, 4.5% w/v, 4.75% w/v, 5% w/v, 5.25% w/v, 5.5% w/v, 5.75% w/v, 6% w/v, 7.25% w/v, 7.5% w/v, 7.75% w/v, 8% w/v, 8.25% w/v, 8.5% w/v, 8.75% w/v, 9% w/v, 10% w/v, 11% w/v, 12% w/v, 13% w/v, 14% w/v, 15% w/v, 16% w/v, 17% w/v, 18% w/v, 19% w/v, or 20% w/v, as well as in a range between any two of said API weight to volume proportions.

In some embodiments the cinchona alkaloid and/or quinoline, e.g., CQ or HCQ, or pharmaceutically acceptable salt thereof, added to a formulation of the present disclosure has an initial purity of at least 90% w/w, for example at least: 90% w/w, 91% w/w, 92%, 93% w/w, 94% w/w, 95% w/w, 96% w/w, 97% w/w, 98% w/w, or 99% w/w.

In some embodiments, formulations of the disclosure have a proportion of the overall amount of the cinchona alkaloid and/or quinoline, e.g., CQ or HCQ, or pharmaceutically acceptable salt thereof, present in the formulation that is dissolved in the liquid phase (i.e., in solution) that is 70%, 75%, 80%, 85%, 90%, 91%, 92%, 93%, 94%, 95%, 96%, 97%, 98%, 99%, 100%, or in a range between any two of such solution proportions.

In some embodiments, formulations of the disclosure contain a solvent. Solvents useful in such formulations include glycerin (glycerol), propylene glycol, a polyethylene glycol (e.g. PEG 400 or PEG 600), and combinations thereof. Such solvents may be included in formulations of the disclosure in weight to volume proportions of 20% w/v, 25% w/v, 30% w/v, 35% w/v, 40% w/v, 45% w/v, 50% w/v, 55% w/v, 60% w/v, 65% w/v, 70% w/v 75% w/v, 80% w/v, 85% w/v, 90% w/v, 95% w/v, 100% w/v, 105% w/v, 110% w/v, 115% w/v, 120% w/v, 125% w/v or in a range between any two of said solvent proportions. The formulations may comprise combinations of solvents, in amounts that individually or in aggregate achieve(s) the stated weight to volume proportions. Such solvents may be included in formulations of the disclosure in concentrations of from 462.5 mg/ml to 1260 mg/ml, and particular concentrations of solvents useful in formulations of the disclosure include 462.5 mg/ml, 475 mg/ml, 500 mg/ml, 525 mg/ml, 550 mg/ml, 575 mg/ml, 600 mg/ml, 625 mg/ml, 650 mg/ml, 675 mg/ml, 700 mg/ml, 725 mg/ml, 750 mg/ml, 775 mg/ml, 800 mg/ml, 825 mg/ml, 850 mg/ml, 875 mg/ml, 900 mg/ml, 925 mg/ml, 950 mg/ml, 975 mg/ml, 1000 mg/ml, 1025 mg/ml, 1050 mg/ml, 1075 mg/ml, 1100 mg/ml, 1125 mg/ml, 1150 mg/ml, 1175 mg/ml, 1200 mg/ml, 1225 mg/ml, 1250 mg/ml, 1,260 mg/ml, or in a range between any two of said solvent concentrations. The formulations may comprise combinations of solvents, in amounts that individually or in aggregate achieve(s) the stated weight to volume concentrations.

In some embodiments, a formulation of the disclosure can consist of the cinchona alkaloid or the quinoline, such as CQ and/or HCQ, as the API, glycerine or a polyethylene glycol as the solvent. In some embodiments, a formulation of the disclosure can consist of the cinchona alkaloid or a quinoline, such as, CQ and/or HCQ, as the API, glycerine or polyethylene glycol as the solvent, and a carbonate as described below. In some embodiments, a formulation of the disclosure can consist of the cinchona alkaloid or the quinoline, such as, CQ and/or HCQ as the API, glycerine or polyethylene glycol as the solvent, and a carbonate as described below and a complexing agent as described below. In some embodiments, a formulation of the disclosure can consist of the cinchona alkaloid or the quinoline, such as CQ and/or HCQ, as the API, glycerine or polyethylene glycol as the solvent, and a carbonate as described below, a complexing agent as described below and a pH adjusting agent as described below. In any such embodiments, the pH of the formulation can be from 6 to 11 as described below. In some embodiments, a formulation of the disclosure can consist of the cinchona alkaloid or the quinoline, such as CQ and/or HCQ as the API, glycerine or polyethylene glycol as the solvent, and a carbonate as described below, a complexing agent as described below and a pH adjusting agent as described below and one or more of a sweetener, flavorant, polymer, surfactant, tonicity agent and preservative as described below.

In some embodiments, formulations of the disclosure contain a carbonate or a bicarbonate. Some carbonates and bicarbonates useful in the formulations of the present disclosure include sodium carbonate, potassium carbonate, calcium carbonate, magnesium carbonate, ammonium carbonate, sodium bicarbonate, potassium bicarbonate, calcium bicarbonate, magnesium bicarbonate, ammonium bicarbonate, or a combination thereof. In such embodiments, formulations of the disclosure may comprise carbonates or bicarbonates in concentrations of from 0.001 mg/ml to 5 mg/ml, and particular concentrations of carbonates or bicarbonates useful in formulations of the disclosure include 0.001 mg/ml, 0.0025 mg/ml, 0.005 mg/ml, 0.0075 mg/ml, 0.01 mg/ml, 0.025 mg/ml, 0.05 mg/ml, 0.075 mg/ml, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1 mg/ml, 1.5 mg/ml, 2 mg/ml, 2.5 mg/ml, 3 mg/ml, 3.5 mg/ml, 4 mg/ml, 4.5 mg/ml, and 5 mg/ml, as well as in a range between any two of said carbonate or bicarbonate concentrations. The formulations may comprise combinations of carbonate or bicarbonate, in amounts that individually or in aggregate achieve(s) the stated carbonate or bicarbonate concentrations.

In some embodiments, formulations of the disclosure contain divalent ion chelating agents such as, without limitation, ethylenediaminetetraacetic acid (“EDTA”), ethylene glycol tetraacetic acid (“EGTA”), ethylenediamine-N,N′-disuccinic acid (“EDDS”), or a combination thereof. The divalent chelating agent may be present in liquid pharmaceutical formulations of the disclosure in weight to volume proportions of from 0.5% w/v to 10% w/v, and particular proportions include 0.01% w/v, 0.05% w/v, 0.075% w/v, 0.1% w/v, 0.5% w/v, 0.75% w/v, 1% w/v, 1.5% w/v, 2% w/v, 2.5% w/v, 3% w/v, 3.5% w/v, 4% w/v, 4.5% w/v, 5% w/v, 5.5% w/v, 6% w/v, 7.5% w/v, 8% w/v, 8.5% w/v, 9% w/v, 9.5% w/v, 10% w/v, as well as in a range between any two of said divalent chelating agent proportions. The formulations may comprise combinations of divalent chelating agents in amounts that individually or in aggregate achieve(s) the stated weight to volume proportions.

In some embodiments, formulations of the disclosure can include a sweetener. Sweeteners useful in the formulations of the present disclosure include acesulfame-K, advantame, alitame, aspartame, brazzein, carrelame, curculin, cyclamic acid, corn syrup (e.g., high fructose corn syrup), cyclamate, dihydrochalchone, erythritol, fructose, galactose, glucose, glycerin, glycine, glycyrrhizic acid, hydrogenated glucose syrup, hydrogenated starch hydrolysate, isomalt, lactitol, lactose, mabilin, miraculin, maltitol, maltodextrin, maltose, monatin, mannitol, mannose, mogrosides, monellin, neohesperidin, pentadin, saccharin, sorbitol, stevia glycosides, sucralose, sucrose, tagatose, tryptophan, and xylitol. The sweetener may be present in liquid pharmaceutical formulations of the disclosure in weight to volume proportions of 0.10% w/v, 0.15% w/v, 0.20% w/v, 0.25% w/v, 0.30% w/v, 0.35% w/v, 0.40% w/v, 0.45% w/v, 0.50% w/v, 0.55% w/v, 0.60% w/v, 0.65% w/v, 0.70% w/v 0.75% w/v, 0.80% w/v, 80.5% w/v, 0.90% w/v, 0.95% w/v, 1% w/v, 2% w/v, 3% w/v, 4% w/v, 5% w/v, 6% w/v, 7% w/v, 8% w/v, 9% w/v, 10% w/v or in a range between any two of said sweetener proportions. The formulations may comprise combinations of sweeteners, in amounts that individually or in aggregate achieve(s) the stated weight to volume proportions.

In some embodiments, formulations of the disclosure can include a flavorant. Flavorants useful in the formulations of the present disclosure include chocolate, vanilla, caramel, orange, lemon, lime, strawberry, raspberry, blueberry, cherry, cinnamon, and nutmeg. The flavorant may be present in liquid pharmaceutical formulations of the disclosure in weight to volume proportions of 0.10% w/v, 0.15% w/v, 0.20% w/v, 0.25% w/v, 0.30% w/v, 0.35% w/v, 0.40% w/v, 0.45% w/v, 0.50% w/v, 0.55% w/v, 0.60% w/v, 0.65% w/v, 0.70% w/v 0.75% w/v, 0.80% w/v, 80.5% w/v, 0.90% w/v, 0.95% w/v, 1% w/v, 2% w/v, 3% w/v, 4% w/v, 5% w/v, 6% w/v, 7% w/v, 8% w/v, 9% w/v, 10% w/v or in a range between any two of said flavorant proportions. The formulations may comprise combinations of flavorant, in amounts that individually or in aggregate achieve(s) the stated weight to volume proportions

In some embodiments, formulations of the disclosure can contain an additional pH adjusting agent and/or a buffer. Additional acidic pH adjusting agents useful in formulations of the disclosure include fumaric acid, formic acid, acetic acid, trichloroacetic acid, benzoic acid, oxalic acid, hydrofluoric acid, hydrogen sulfide, nitrous acid, sulfurous acid, phosphoric acid, and combinations thereof. Additional alkaline pH adjusting useful in formulations of the disclosure include sodium hydroxide, potassium hydroxide, calcium hydroxide, ammonium hydroxide, ethanolamine, and trolamine. Additional buffers useful in formulations of the disclosure include is acetic acid, sodium acetate, benzoic acid, sodium benzoate, boric acid, sodium borate, citric acid, sodium citrate, sodium phosphate, monobasic sodium phosphate, dibasic sodium phosphate, potassium phosphate, monobasic potassium phosphate, dibasic potassium phosphate, sodium acetate, lactic acid, a tartaric acid, sodium tartrate, tris(hydroxymethyl)aminomethane (“TRIS”), or a combination thereof. In such formulations, the additional buffer and/or pH adjusting agent are present in the formulations in amounts, alone or together, that are sufficient to cause the formulation to have a pH of from 6 to 11, for example pH 6, pH 6.1, pH 6.2, pH 6.3, pH 6.4, t pH 6.5, pH 6.6, pH 6.7, pH 6.8, pH 6.9, pH 7, pH 7.1, pH 7.2, pH 7.3, pH 7.4, pH 7.5, pH 7.6, pH 7.7, pH 7.8, pH 7.9, pH 8, pH 8.1, pH 8.2, pH 8.3, pH 8.4, pH 8.5, pH 8.6, pH 8.7, pH 8.8, pH 8.9, pH 9, pH 9.1, pH 9.2, pH 9.3, pH 9.4, pH 9.5, pH 9.6, pH 9.7, pH 9.8, pH 9.9, pH 10, pH 9, pH 9.1, pH 9.2, pH 9.3, pH 9.4, pH 9.5, pH 9.6, pH 9.7, pH 9.8, pH 9.9, pH 10, pH 10.1, pH 10.2, pH 10.3, pH 10.4, pH 10.5, pH 10.6, pH 10.7, pH 10.8, pH 10.9, pH 11, as well as in a range between any two such pH values.

In some embodiments, formulations of the present disclosure are pourable. The viscosities of such formulations can range from 1 centipoise (“cps”) (i.e., the viscosity of water at room temperature) to 25,000 cps (i.e., the viscosity of chocolate syrup at room temperature); and exemplary particular viscosities of formulations of the disclosure include 1 cps, 25 cps, 50 cps, 75 cps, 100 cps, 150 cps, 200 cps (about the viscosity of maple syrup at room temperature), 250 cps, 300 cps, 400 cps, 500 cps, 600 cps, 700 cps, 800 cps, 900 cps, 1000 cps (about the viscosity of glycerin at room temperature), 1100 cps, 1200 cps, 1300 cps, 1400 cps, 1500 cps, 1600 cps, 1700 cps, 1800 cps, 1900 cps, 2000 cps, 2100 cps, 2200 cps, 2300 cps, 2400 cps, 2500 cps, 2600 cps, 2700 cps, 2800 cps, 2900 cps, 3000, 3500 cps, 4000 cps, 4500 cps, 5000 cps, 6000 cps, 7000 cps, 8000 cps, 9000 cps, 10,000 cps, 12,500 cps, 15,000 cps, 17,500 cps, 20,000, cps 22,500 cps, 25,000 cps (about the viscosity of chocolate syrup at room temperature), 27,500 cps, 30,000, cps as well as in a range between any two of said viscosities.

In some embodiments, formulations of the disclosure can contain a polymer. Non-ionic polymers useful in certain formulations of the disclosure include hydroxyethyl cellulose, hydroxypropylmethyl cellulose, methyl cellulose, carboxymethyl cellulose, polyvinyl pyrrolidone, and polyvinyl alcohol. Ionic polymers useful in certain formulations of the disclosure include polyacrylates (e.g., carbopols and carbomers), alginates, chitosans, hyaluronic acid, and xanthan gum. Such ionic and/or nonionic polymers may be present in formulations of the disclosure in weight to volume proportions of the overall formulation of 0.001% w/v, 0.005% w/v, 0.01% w/v, 0.05% w/v, 0.1% w/v, 0.25% w/v, 0.5% w/v, 0.75% w/v, 1.0% w/v, 1.25% w/v, 1.5% w/v, 1.75% w/v, 2.0% w/v, 2.25% w/v, 2.5% w/v, 2.75% w/v, 3.0% w/v, 3.25% w/v, 3.5% w/v 3.75% w/v, 4.0% w/v, 4.25% w/v, 4.5% w/v, 4.75% w/v, or 5.0% w/v, as well as in a range between any two of said polymer proportions. The formulations may comprise combinations of polymers, in amounts that individually or in aggregate achieve(s) the stated polymer proportions.

In some embodiments, formulations of the disclosure contain a surfactant. Surfactants useful in certain formulations of the disclosure include sodium lauryl sulfate, docusate sodium, phosphatidylcholine, lecithin, betaines, tyloxapol, polyoxyethylene sorbitan esters, such as polysorbate 20, polysorbate 60, and polysorbate 80; polyethoxylated castor oils, such as cremaphor, polyethoxylated hydrogenated castor oils, such as HCO-40; and poloxamers. Such surfactants may be present in formulations of the disclosure in weight to volume proportions of the overall formulation of 0.001% w/v, 0.005% w/v, 0.01% w/v, 0.05% w/v, 0.1% w/v, 0.2% w/v, 0.3% w/v, 0.4% w/v, 0.5% w/v, 0.6% w/v, 0.7% w/v, 0.8% w/v, 0.9% w/v, 1.0% w/v, 1.1% w/v, 1.2% w/v, 1.3% w/v, 1.4% w/v, 1.5% w/v, 1.6% w/v, 1.7% w/v, 1.8% w/v, 1.9% w/v, 2.0% w/v, or in a ranges between any two of said surfactant proportions. The formulations may comprise combinations of surfactants, in amounts that individually or in aggregate achieve(s) the stated weight to volume proportions.

In some embodiments, formulations of the disclosure can contain a tonicity agent. Ionic tonicity agents useful in certain formulations of the disclosure include calcium chloride, magnesium chloride, potassium chloride, sodium chloride, sodium sulfate, and combinations thereof. Nonionic tonicity agents useful in the formulations described herein include mannitol, sorbitol, xylitol, erythritol, lactitol, maltitol, isomalt, and combinations thereof. The formulations may comprise tonicity agent in weight to volume proportions of the overall formulation of 0.001% w/v, 0.005% w/v, 0.01% w/v, 0.05% w/v, 0.1% w/v, 0.2% w/v, 0.3% w/v, 0.4% w/v, 0.5% w/v, 0.6% w/v, 0.7% w/v, 0.8% w/v, 0.9% w/v, 1.0% w/v, 1.1% w/v, 1.2% w/v, 1.3% w/v, 1.4% w/v, 1.5% w/v, 1.6% w/v, 1.7% w/v, 1.8% w/v, 1.9% w/v, 2.0% w/v, or in a range between any two of said tonicity agent proportions. The formulations may comprise combinations of tonicity agent, in amounts that individually or in aggregate achieve(s) the stated tonicity weight to volume proportions. In some embodiments, formulations of the disclosure can contain a preservative. Preservatives useful in certain formulations of the disclosure include dibutylhydroxytoluene, benzalkonium chloride, benzyl alcohol, borates, parabens, cresols, benzoic acid, phenol, sorbic acid, benzethonium chloride, sodium chlorite and combinations thereof. The formulations may comprise preservative in weight to volume proportions of the overall formulation of 0.001% w/v, 0.005% w/v, 0.01% w/v, 0.05% w/v, 0.1% w/v, 0.25% w/v, 0.5% w/v, 0.75% w/v, 1.0% w/v, 1.25% w/v, 1.5% w/v, 1.75% w/v, 2.0% w/v, 2.25% w/v, 2.5% w/v, 2.75% w/v, 3.0% w/v, 3.25% w/v, 3.5% w/v 3.75% w/v, 4.0% w/v, 4.25% w/v, 4.5% w/v, 4.75% w/v, and 5.0% w/v, or in a range between any two of said preservative proportions. The formulations may comprise combinations of preservatives, in amounts that individually or in aggregate achieve(s) the stated weight to volume proportions.

Also disclosed herein is a process of preparing a liquid pharmaceutical formulation that is suitable for oral administration. Such a process can be one comprising:
(i) forming an API-solvent mixture by combining a therapeutically effective amount of an API and an amount of a solvent sufficient to result in from 2 mg/ml to 300 mg/ml of the API being in the solution phase of the liquid formulation, wherein the API is CQ, HCQ, or a combination thereof, or one or more pharmaceutically acceptable salt(s) thereof and wherein the solvent is glycerol, a PEG, or a combination thereof; and
(ii) holding the temperature of the API-solvent mixture at a range of from 35° C. to 290° C., for example from 35° C. to 100° C., from 35° C. to 90° C., from 40° C. to 100° C., from 45° C. to 90° C., from 50° ° C. to 100° C., from 50° ° C. to 90° C. or from 50° C. to 80° C., from 50° C. to 75° C. until from 2 mg/ml to 300 mg/ml of the API is in the solution phase of the mixture; thereby preparing the liquid formulation; and
(iii) cooling, actively or passively, the temperature of the liquid to at 25° C.±2° ° C. at a rate that maintains the 2 mg/ml to 300 mg/ml of the API being in the solution phase of the liquid formulation
wherein the liquid formulation is free of added water.

In such a method, the API can be HCQ sulfate and the solvent can be glycerol.

In any embodiment of such method, the HCQ sulfate can be added in an amount of 40 mg/ml, 45 mg/ml, 50 mg/ml, or 100 mg/ml and glycerol can be added in an amount of 945 mg/ml and 95% or more, for example 96%, 97%, 98% or 99% or more, of the HCQ sulfate can be in the solution phase of the formulation. The amount of the HCQ sulfate in the solution phase of the formulation can range between any two of the above values.

In any embodiment of such methods, the liquid formulation can comprise 40 mg/ml, 45 mg/ml, 50 mg/ml, or 100 mg/ml HCQ sulfate and the balance glycerol; and the solution phase of the liquid formulation can include 95% or more of the HCQ sulfate, for example 96%, 97%, 98% or 99% or more, of the HCQ sulfate can be in the solution phase of the formulation. The amount of the HCQ sulfate in the solution phase of the formulation can range between any two of the above values.

### SOME EXEMPLARY EMBODIMENTS

Some specific, non-limiting embodiments within the present disclosure are enumerated below. This listing of particular embodiments is not intended to limit the scope of the disclosure.

Embodiment 1: A liquid pharmaceutical formulation, suitable for oral administration, that comprises:
a therapeutically effective amount of an active pharmaceutical ingredient (API) that is one or more of chloroquine (CQ), hydroxychloroquine (HCQ), or a combination thereof, or one or more pharmaceutically acceptable salt(s) thereof; and
an amount of a solvent sufficient to result in from 2 mg/ml to 300 mg/ml of the API being in the solution phase of the formulation;
wherein the solvent is glycerol, a polyethylene glycol (PEG), or a combination thereof and wherein the liquid formulation is free of added water.

Embodiment 2: The formulation of Embodiment 1, wherein the API is HCQ sulfate, and wherein from 10 mg/ml to 275 mg/ml of the HCQ sulfate is in the solution phase of the formulation.

Embodiment 3: The formulation of Embodiment 1, wherein the API is HCQ sulfate, and wherein from 25 mg/ml to 275 mg/ml of the HCQ sulfate is in the solution phase of the formulation.

Embodiment 4: The formulation of Embodiment 1, wherein the API is HCQ sulfate, and wherein from 40 mg/ml to 50 mg/ml of the HCQ sulfate is in the solution phase of the formulation.

Embodiment 5: The formulation of Embodiment 2, wherein the amount of the solvent is from 462.5 mg/ml to 1260 mg/ml, and wherein the solvent is glycerol.

Embodiment 6: The formulation of Embodiment 3, wherein the amount of the solvent is from 630 mg/ml to 1260 mg/ml, and wherein the solvent is glycerol.

Embodiment 7: The formulation of Embodiment 4, wherein the amount of the solvent is from 945 mg/ml to 1260 mg/ml, and wherein the solvent is glycerol.

Embodiment 8: The formulation of Embodiment 4, wherein the amount of the solvent is 1260 mg/ml, and wherein the solvent is glycerol.

Embodiment 9: A method of treating uncomplicated malaria due to Plasmodium falciparum, rheumatoid arthritis, lupus erythematosus, or chronic discoid lupus erythematosus, comprising orally administering the formulation of any one of Embodiments 1 to 8 to a subject presenting treating uncomplicated malaria due to Plasmodium falciparum, rheumatoid arthritis, lupus erythematosus, or chronic discoid lupus erythematosus.

Embodiment 10: The method of Embodiment 9, wherein the formulation is administered to the subject once daily and wherein the formulation comprises from 40 mg/ml to 50 mg/ml of the chloroquine, HCQ, or pharmaceutically acceptable salt(s) thereof of.

Embodiment 11: A liquid pharmaceutical formulation, suitable for oral administration, that comprises:
40 mg/ml, 45 mg/ml, 50 mg/ml, or 100 mg/ml HCQ sulfate; and
at least 945 mg/ml glycerol;
   wherein 95% or more of the HCQ sulfate is in the solution phase of the formulation, and wherein the liquid formulation is free of added water.

Embodiment 12: The formulation of Embodiment 11, wherein the formulation comprises the HCQ sulfate and balance glycerol.

Embodiment 13: A method of treating uncomplicated malaria due to Plasmodium falciparum, rheumatoid arthritis, lupus erythematosus, or chronic discoid lupus erythematosus, comprising orally administering the formulation of Embodiment 11 or 12 to a subject presenting treating uncomplicated malaria due to Plasmodium falciparum, rheumatoid arthritis, lupus erythematosus, and chronic discoid lupus erythematosus.

Embodiment 14: The method of Embodiment 13, wherein the formulation is administered to the subject once daily and wherein the formulation comprises from 40 mg/ml to 50 mg/ml of the HCQ sulfate.

Embodiment 15: A process of preparing a liquid pharmaceutical formulation that is suitable for oral administration, said process comprising:
(i) forming an API-solvent mixture by combining a therapeutically effective amount of an API and an amount of a solvent sufficient to result in from 2 mg/ml to 300 mg/ml of the API being in the solution phase of the liquid formulation, wherein the API is CQ, HCQ, or a combination thereof, or one or more pharmaceutically acceptable salt(s) thereof and wherein the solvent is glycerol, a PEG, or a combination thereof; and
(ii) holding the temperature of the API-solvent mixture at a range of from 25° ° C. to 290° ° C., or at a temperature of 20° C., 25° C., 30° C., 35° C., 40° ° C., 45° C., 50° C., 55° C., 60° C., 65° C., 70° C., 75° C., 80° C., 85° C., 90° C., 95° C., 100° C., 110° C., 125° C., 150° C., 175° C., 200° ° C., 150° C., 200° C., 150° C., 275° C., or 100° C., or range between any two of the stated temperatures until from 2 mg/ml to 300 mg/ml of the API is in the solution phase of the mixture and thereby preparing the liquid formulation; and
(iii) cooling, actively or passively, the temperature of the liquid to at 25° C.±2° C. at a rate that maintains the 2 mg/ml to 300 mg/ml of the API being in the solution phase of the liquid formulation
wherein the liquid formulation is free of added water.

Embodiment 16: The method of Embodiment 15, wherein the API is HCQ sulfate and wherein the solvent is glycerol.

The method of Embodiment 15 or 16, wherein the liquid formulation comprises the HCQ sulfate in an amount of 40 mg/ml, 45 mg/ml, 50 mg/ml, or 100 mg/ml and the glycerol in an amount of at 945 mg/ml and wherein 95% or more of the HCQ sulfate is in the solution phase of the formulation.

The method of Embodiment 15 or 16, wherein the liquid formulation comprises 40 mg/ml, 45 mg/ml, 50 mg/ml, or 100 mg/ml HCQ sulfate and balance glycerol and wherein wherein 95% or more of the HCQ sulfate is in the solution phase of the liquid formulation.

### EXAMPLES

Aspects of embodiments of the present disclosure may be further understood in light of the following examples, which should not be construed as limiting in any way. In the experiments of the Examples, solubility of HCQ (as HCQ sulfate) in liquid formulation was studied.

### Example 1

HCQ sulfate solubility. Liquid HCQ sulfate formulations 266-28A and 266-28B comprised the ingredients set forth in Table 1.1 and were studied in the HCQ sulfate solubility experimental protocols described in this Example 1.

TABLE 1.1
HCQ sulfate formulations 266-28A andB

| | 266-28A | 266-28B |
|---|---|---|
| Ingredient | mg/50 ml | mg/50 ml |
| Hydroxychloroquine sulfate | 20,000 | 20,000 |
| Glycerin | 63,000 | 63,000 |
| Total | 83,000 | 83,000 |

Liquid HCQ formulations. Formulations 266-28A and 266-28B were made as follows. Step 1: 63 g of glycerol were weighed and transferred into a 50 ml beaker with magnetic stir bar and heated to 70° C. while stirred. Step 2: 10 g HCQ sulfate were weighed and slowly added to the 70° C. glycerol of Step 1 with continued stirring and with maintenance of 70° C. until a clear liquid solution was formed. Step 3: 10 g HCQ sulfate was weighed and slowly added to resultant clear liquid solution of Step 3 with continued stirring and with maintenance of 70° C. until a clear liquid solution was formed. Step 4: the resultant clear liquid solution of Step 4 was cooled to room temperature. Step 5: the beaker containing the resultant room temperature liquid from Step 4 was sealed with parafilm and mixed at room temperature for 24 hours and then the liquid was transferred to the analytical research department for HCQ sulfate solubility studies, while being kept at 25° C.±2° C.

The HPLC assay for HCQ. The reference standards, reagents, and solutions used in the high-performance liquid chromatography (“HPLC”) analytical methods for HCQ sulfate of the present disclosure were as set forth in Table 1.2.

TABLE 1.2
HCQ assay and related substance assay reference standards, reagents, and solutions

| | |
|---|---|
| HCQ sulfate USP | Reference standard or secondary reference standard |
| HCQ-N-oxide | Standard |
| Acetonitrile | HPLC grade |
| Potassium | Analytical reagent grade |
| dihydrogen | |
| orthophosphate | |
| Orthophosphoric | Analytical reagent grade |
| acid | |
| Water | Purified water |
| Buffer solution | 6.8 g of potassium dihydrogen orthophosphate was weighed and transferred |
| | into a 1000 ml volumetric flask and dissolved and diluted to volume with |
| | purified water. The resultant solution was adjusted to pH 2.4 with |
| | orthophosphoric acid. |
| Mobile Phase A | A degassed mixture of buffer solution and acetonitrile (95:5 v/v) was |
| | prepared. |
| Mobile Phase B | A degassed mixture of buffer solution and acetonitrile (30:70 v/v) was |
| | prepared. |
| Diluent | Same as mobile phase A. |
| Resolution stock | 2 mg of HCQ-N-oxide standard was weighed and transferred into a 200 ml |
| solution | volumetric flask and then 160 ml of diluent was added and the resultant |
| | solution was sonicated until the HCQ-N-oxides was dissolved and diluted to |
| | 200 ml with diluent and mixed well. |
| Resolution solution | 20.0 mg of HCQ sulfate standard was weighted and transferred into a 200 ml |
| | volumetric flask and then 160 ml of diluent was added. The resultant solution |
| | was sonicated until the HCQ was dissolved, then 3.0 ml of resolution stock |
| | solution was added and diluted to 200 ml with the diluent and mixed well. The |
| | HCQ Sulfate concentration was 0.1 mg/ml and the HCQ-N-oxide |
| | concentration was 15 μg/ml of the diluent. |
| Standard stock | 50.0 mg of HCQ sulfate standard was weighted and transferred into a 50 ml |
| solution | volumetric flask and 30 ml of diluent was added. The resultant solution was |
| | sonicated until dissolved and then diluted to 50 ml with diluent and mixed |
| | well. |
| Standard solution | 10.0 ml of standard stock solution was transferred into a 100 ml volumetric |
| | flask, diluted to 10 ml, and mixed well. The HCQ sulfate concentration was |
| | 0.1 mg/ml. |
| Standard check | Prepared the same as standard stock solution. |
| stock solution | |
| Standard check | 10.0 ml of standard check stock solution was transferred into a 100 ml |
| solution | volumetric flask, diluted to 100 ml, and mixed well. The HCQ sulfate |
| | concentration was 0.1 mg/ml. |
| Sensitivity solution | 1.0 ml of standard solution was transferred into a 10 ml volumetric flask and |
| | diluted to 10 ml with diluent and then mixed well. 1.0 ml of the resultant |
| | solution was transferred into a 100 ml volumetric flask, diluted to 100 ml with |
| | diluent and mixed well. The HCQ sulfate concentration was 0.1 μg/ml |
| Sample Solution | Based on the target concentration of the sample, serial dilution in diluent were |
| (In-process and | prepared to achieve solutions that contained about 0.05 mg to 0.15 mg HCQ |
| Finished product) | sulfate. |

The high performance liquid chromatography (“HPLC”) conditions for the HCQ assay and related substances assay employed in the present disclosure were according to those set forth in Table 1.3.

TABLE 1.3
HCQ assay and substance HPLC chromatographic parameters

Chromatographic

| parameters | Equipment and/or conditions | |
|---|---|---|
| System | HPLC system equipped with, Binary | |
| | gradient and UV detector | |
| Column | YMC pack Pro C18, ( 250 × 4.6 ) mm, 5 μm | |
| Column Temperature | 40° C. ± 2° C. | |
| Sample Tray Temperature | 25° | C. |
| Detector Wavelength | UV 220 nm | |
| Pump Mode | Gradient | |
| Flow Rate | 1.7 | ml/min |
| Injection Volume | 20 | μl |
| HCQ sulfate retention time | Between 6.0 and 8.5 minutes | |
| Run Time | 45 | minutes |

The HPLC gradient elution program used in the HCQ assay was as set forth in Table 1.4.

TABLE 1.4
HPLC gradient elution program for
HCQ assay and related substances

| Time in (Min) | Mobile phase A (% v/v) | Mobile phase B (% v/v) |
|---|---|---|
| 0 | 93 | 7 |
| 10 | 93 | 7 |
| 25 | 65 | 35 |
| 35 | 65 | 35 |
| 37 | 93 | 7 |
| 45 | 93 | 7 |

HPLC steps. Step 1. The HPLC system was equilibrated with mobile phase A for about 30 minutes. Iterative injections of diluent were made until a clean and reproducible baseline was achieved. The chromatogram was recorded and used to identify any peak eluting at the retention time of major peaks. Step 2. One injection of sensitivity solution into the HPLC column was performed. The chromatogram was recorded and used to identify the HCQ peak and calculate its signal to noise ratio. Step 3. One injection of resolution solution into the HPLC column was performed. The chromatogram was recorded and used to identify HCQ and HCQ-n-oxide peaks and the resolution between those peaks was calculated. Step 4. Six replicate injections of standard solution into the HPLC column were performed. The chromatograms were recorded and used to calculate the average and % RSD for the HCQ peak area responses obtained from the six replicate injections of standard solution. The tailing factor was determined. Step 5. Two replicate injections of standard check solution into the HPLC column were performed. The chromatograms were recorded and used to calculate average peak area responses of the HCQ obtained from the two replicate injections of standard check solution. The similarity factor was calculated. Step 6. One injection of diluent into the HPLC column was performed before injecting sample solutions. Step 7. One injection of sample solution into the HPLC column was made for six independent samples. The chromatograms for each of the six injected samples were recorded and used to determine the peak area of HCQ. The HCQ Hydroxychloroquine concentration in the sample solution was calculated. Steps 8 and 10. After every six injections of sample solution and at the end of the sequence, one injection of diluent into the HPLC column was performed. Step 9. One injection of standard solution into the HPLC column was made (bracketing). The chromatogram was recorded and used to determine peak area of HCQ in standard solution (bracketing) after every six injections. The % RSD was calculated of HCQ peak area obtained from the initial 6 injections of standard solution and bracketing standard.

The equation employed to calculate the similarity factor in the HCQ and related substances assay were as set forth in Table 1.5.

TABLE 1.5
HCQ assay and related substances similarity factor equation
$Similarity factor = \frac{Acstd}{Awstd} \times \frac{Wwstd}{Wcstd} \times 100$

| | |
|---|---|
| Where: | |
| Acstd = | peak area of the check standard |
| Awstd = | peak area of the working standard |
| Wwstd = | weight of the working standard |
| Wcstd = | weight of the check standard |

The equations employed to calculate the percent (%) assay in mg/ml (formulation) and percent (%) known and unknown impurities in the HCQ and related substances assay were as set forth in Table 1.6.

TABLE 1.6
HCQ assay (in percent (%) and in mg/ml) and
related substances (percent (%) known and unknown impurities) equations
$\% Assay = \frac{A spl}{Astd} \times \frac{Wstd}{50 mL} \times \frac{P}{100 \%} \times \frac{10 mL}{100 mL} \times \frac{Wspl}{50 mL} \times \frac{100 mL}{10 mL} \times 100 \%$
$Assay ( mg / mL ) = \frac{A spl}{Astd} \times \frac{Wstd}{50 mL} \times \frac{P}{100 \%} \times \frac{10 mL}{100 mL} \times \frac{V 1 spl}{VD 1} \times \frac{V 2 spl}{VD 2} \times 100 \%$
$\% Impurity = \frac{A imp}{Astd} \times \frac{Wstd}{50 mL} \times \frac{P}{100 \%} \times \frac{10 mL}{100 mL} \times \frac{Wspl}{50 mL} \times \frac{100 mL}{10 mL} \times CF \times 100 \%$

| | |
|---|---|
| Where: | |
| A spl = | Peak area of Hydroxychloroquine obtained from the sample solution |
| A imp = | Peak area of Individual impurity obtained from the sample solution |
| Astd = | Peak area of the Hydroxychloroquine Sulfate obtained from standard solution |
| Wstd = | Weight of Reference standard used to prepare standard solution |
| Wspl = | Weight of API used to prepare sample solution |
| P = | Potency/assay of Hydroxychloroquine Sulfate standard in percentage (on as is basis) |
| VD1sp1 = | Volume of formulation sample used for first dilution |
| V1spl = | Volume of volumetric flask used for first dilution |
| VD2spl = | Volume of first dilution used for second dilution |
| V2spl = | Volume of volumetric flask used for second dilution |
| CF = | correction factor of respective impurity |

The relative retention time (“RRT”) and correction factor (“CF”) of impurities in the HCQ assay were as set forth in Table 1.7.

TABLE 1.7
RRT and CF of impurities in the HCQ assay and related substances

| Impurity | RRT | CF |
|---|---|---|
| Desethyl Hydroxychloroquine | 0.84 | 0.77 |
| Hydroxychloroquine-N-oxide | 1.30 | 0.89 |
| Hydroxychloroquine-O-acetate | 1.61 | 0.89 |
| Hydroxychloroquine-O-sulfate | 1.99 | 1.05 |
| Any unspecified impurity | Report | 1.00 |

Limit of quantification (“LOQ”) values of HCQ sulfate and known impurities in the HCQ and related substances assay were as set forth in Table 1.8.

TABLE 1.8
LOQ values of HCQ sulfate and known impurities
in the HCQ assay and related substances

| Components | LOQ (%) |
|---|---|
| Hydroxychloroquine Sulfate | 0.03 |
| Desethyl Hydroxychloroquine | 0.02 |
| Hydroxychloroquine-N-oxide | 0.03 |
| Hydroxychloroquine-O-acetate | 0.03 |
| Hydroxychloroquine-O-sulfate | 0.04 |

The results of the HCQ assay analysis for the solubility experiments conducted on the 266-28A and 266-28B formulations described above are reported in Table 1.9.

TABLE 1.9
Solubility of HCQ sulfate in formulations 266-28A-B

| | 266-28A | 266-28B |
|---|---|---|
| HCQ solubility | 273.3 mg/ml | 273.1 mg/ml |

The result of this Example shows that the dissolution protocol above for preparing a liquid formulation can be used to dissolve 273 mg/ml of HCQ in glycerol.

While the foregoing specification teaches the principles of the present invention, with examples provided for the purpose of illustration, it will be understood that the practice of the invention encompasses all variations, adaptations and/or modifications as come within the scope of the following claims and their equivalents.

1.-10. (canceled)

11. A liquid pharmaceutical formulation, suitable for oral administration, that per each milliliter (“ml”) comprises:
a hydroxychloroquine (“HCQ”) solubility vehicle that consists of 35 mg to 85 mg HCQ, or a pharmaceutically acceptable salt thereof, and 1,175 mg to 1225 mg glycerol;
wherein 95% or more of the HCQ is in the solution phase of the formulation, and wherein the formulation is free of any added HCQ solubilizer of other than the glycerol of the vehicle.

12. (canceled)

13. A method of treating uncomplicated malaria due to Plasmodium falciparum, rheumatoid arthritis, lupus erythematosus, or chronic discoid lupus erythematosus, comprising orally administering the formulation of claim 11 to a subject presenting treating uncomplicated malaria due to Plasmodium falciparum, rheumatoid arthritis, lupus erythematosus, and chronic discoid lupus erythematosus.

14. The method of claim 13, wherein the formulation is administered to the subject once daily and wherein the formulation comprises from 40 mg/ml to 50 mg/ml of the HCQ sulfate.

15. A process of preparing a liquid pharmaceutical formulation that is suitable for oral administration, said process comprising:
(i) forming an API-solvent mixture by combining a therapeutically effective amount of an API and an amount of a solvent sufficient to result in from 2 mg/ml to 300 mg/ml of the API being in the solution phase of the liquid formulation, wherein the API is CQ, HCQ, or a combination thereof, or one or more pharmaceutically acceptable salt(s) thereof and wherein the solvent is glycerol; and
(ii) holding the temperature of the API-solvent mixture at a range of from 25° C. to 290° C., or at a temperature of 20° C., 25° C., 30° C., 35° C., 40° ° C., 45° C., 50° C., 55° C., 60° C., 65° C., 70° C., 75° C., 80° C., 85° C., 90° C., 95° C., 100° C., 110° C., 125° C., 150° C., 175° C., 200° C., 150° C., 200° C., 150° C., 275° C., or 100° C., or range between any two of the stated temperatures until from 2 mg/ml to 300 mg/ml of the API is in the solution phase of the mixture and thereby preparing the liquid formulation; and
(iii) cooling, actively or passively, the temperature of the liquid to at 25° C.±2° C. at a rate that maintains the 2 mg/ml to 300 mg/ml of the API being in the solution phase of the liquid formulation
wherein the liquid formulation is free of added water.

16. The method of claim 15, wherein the API is HCQ sulfate.

17. The method of claim 16, wherein the liquid formulation comprises the HCQ sulfate in an amount of 40 mg/ml, 45 mg/ml, 50 mg/ml, or 100 mg/ml and the glycerol in an amount of at 945 mg/ml and wherein 95% or more of the HCQ sulfate is in the solution phase of the formulation.

18. The method of claim 16, wherein the liquid formulation comprises 40 mg/ml, 45 mg/ml, 50 mg/ml, or 100 mg/ml HCQ sulfate and balance glycerol and wherein 95% or more of the HCQ sulfate is in the solution phase of the liquid formulation.

19. The formulation of claim 11, wherein sulfate is the pharmaceutically acceptable salt of HCQ.

20. A liquid pharmaceutical formulation, suitable for oral administration, that per each milliliter (“ml”) comprises:
an HCQ solubility vehicle that consists of 40 mg to 100 mg HCQ, or a pharmaceutically acceptable salt thereof, and 850 mg to 1220 mg glycerol;
0.1% w/v to 5% w/v of at least one divalent chelating agent;
up to 20% w/v of at least one flavorant;
up to 5% w/v of at least one sweetener; and
up to 1% w/v of at least one preservative,
wherein 95% or more of the HCQ is in the solution phase of the formulation, and wherein the formulation is free of any added HCQ solubilizer of other than the glycerol of the solubility vehicle.

21. The formulation of claim 20, wherein sulfate is the pharmaceutically acceptable salt of HCQ.

22. The formulation of claim 20, wherein the at least one divalent chelating agent is ethylenediaminetetraacetic acid (“EDTA”), ethylene glycol tetraacetic acid (“EGTA”), ethylenediamine-N,N′-disuccinic acid (“EDDS”), or a combination thereof.

23. The formulation of claim 20, wherein the at least one flavorant is chocolate, vanilla, caramel, orange, lemon, lime, strawberry, raspberry, blueberry, cherry, cinnamon, nutmeg, or a combination thereof.

24. The formulation of claim 20, wherein the at least one sweetener is acesulfame-K, advantame, alitame, aspartame, brazzein, carrelame, curculin, cyclamic acid, corn syrup (e.g., high fructose corn syrup), cyclamate, dihydrochalchone, erythritol, fructose, galactose, glucose, glycerin, glycine, glycyrrhizic acid, hydrogenated glucose syrup, hydrogenated starch hydrolysate, isomalt, lactitol, lactose, mabilin, miraculin, maltitol, maltodextrin, maltose, monatin, mannitol, mannose, mogrosides, monellin, neohesperidin, pentadin, saccharin, sorbitol, stevia glycosides, sucralose, sucrose, tagatose, tryptophan, xylitol, or a combination thereof.

25. The formulation of claim 20, wherein the at least one preservative is dibutylhydroxytoluene, benzalkonium chloride, benzyl alcohol, borates, parabens, cresols, benzoic acid, phenol, sorbic acid, benzethonium chloride, sodium chlorite, or a combination thereof.

26. A liquid pharmaceutical formulation, suitable for oral administration, that per each milliliter (“ml”) comprises:
an HCQ solubility vehicle that consists of 40 mg to 100 mg HCQ, or a pharmaceutically acceptable salt thereof, and 850 mg to 1220 mg glycerol;
0.1% w/v to 20% w/v of at least one flavorant;
0.1% w/v to 5% w/v of at least one sweetener;
up to 5% w/v of at least one divalent chelating agent;
up to 1% w/v of at least one preservative, and
wherein 95% or more of the HCQ is in the solution phase of the formulation, and wherein the formulation is free of any added HCQ solubilizer of other than the glycerol of the solubility vehicle.

27. The formulation of claim 26, wherein sulfate is the pharmaceutically acceptable salt of HCQ.

28. The formulation of claim 19, wherein the at least one divalent chelating agent is ethylenediaminetetraacetic acid (“EDTA”), ethylene glycol tetraacetic acid (“EGTA”), ethylenediamine-N,N′-disuccinic acid (“EDDS”), or a combination thereof.

29. The formulation of claim 26, wherein the at least one flavorant is chocolate, vanilla, caramel, orange, lemon, lime, strawberry, raspberry, blueberry, cherry, cinnamon, nutmeg, or a combination thereof.

30. The formulation of claim 26, wherein the at least one sweetener is acesulfame-K, advantame, alitame, aspartame, brazzein, carrelame, curculin, cyclamic acid, corn syrup (e.g., high fructose corn syrup), cyclamate, dihydrochalchone, erythritol, fructose, galactose, glucose, glycerin, glycine, glycyrrhizic acid, hydrogenated glucose syrup, hydrogenated starch hydrolysate, isomalt, lactitol, lactose, mabilin, miraculin, maltitol, maltodextrin, maltose, monatin, mannitol, mannose, mogrosides, monellin, neohesperidin, pentadin, saccharin, sorbitol, stevia glycosides, sucralose, sucrose, tagatose, tryptophan, xylitol, or a combination thereof.

31. The formulation of claim 26, wherein the at least one preservative is dibutylhydroxytoluene, benzalkonium chloride, benzyl alcohol, borates, parabens, cresols, benzoic acid, phenol, sorbic acid, benzethonium chloride, sodium chlorite, or a combination thereof.
